(19) United States
(12) Patent Application Publication
(10) Pub. No.: US 2010/0319725 A1
(43) Pub. Date: Dec. 23, 2010

(54) SYSTEMS, METHODS, AND COMPOSITIONS FOR SANITIZING FOOD PRODUCTS

(75) Inventors: Suraj Puri, Los Altos, CA (US); George Plaut, San Jose, CA (US); Saurabh Sharma, Arlington Heights, IL (US)

Correspondence Address: EcoTech Law Group, P.C., 201 SPEAR STREET, SUITE 1100, SAN FRANCISCO, CA, 94105, US

(73) Assignee: NANO OM TECHNOLOGY, LLC, Los Altos, CA (US)

(21) Appl. No.: 12/487,628

(22) Filed: Jun. 18, 2009

(51) Int. Cl. A23L 1/00; A23L 3/34; B08B 3/12; B08B 3/02; A61K 33/14

(52) U.S. Cl. 134/1; 134/25.3; 134/10; 134/184; 134/109; 134/111; 134/57 R; 424/680

(57) ABSTRACT

Inventive methods, systems and compositions for sanitizing food products are described. A method for sanitizing food products includes: (a) activating a solution, which includes a solute and a solvent, by using acoustic energy to form a coherent solution including solute clusters, in which each solute cluster is organized such that at least one solute molecule is surrounded by many solvent molecules; and (b) submerging the food products into a tank containing the coherent solution to sanitize the food products.

### FIELD OF THE INVENTION

The present invention relates to effectively sanitizing food products using a coherent solution. More particularly, the present invention relates to systems, methods, and compositions for effectively sanitizing food products using a coherent solution which removes harmful microorganisms from food products.

### BACKGROUND OF THE INVENTION

Fresh fruit and vegetable sales make up an important segment of the food economy, and food produce continues to experience increased sales in the United States. The produce supply chain consists of farming, processing, packing, distribution, retail, and food service outlets throughout the country. It has been estimated that sales of fresh produce and other products in the U.S. generate over $275.5 billion in direct economic output. This output, combined with suppliers sales and workers spending generated, in total, an economic impact of $554.4 billion in 2006. This translates to 2.707 million jobs (1.9% of all U.S. employment) and accounts for 4.23% of U.S. GDP and one third of total U.S. animal and crop production. Total combined retail and food services fresh produce sales were $94.8 billion in 2005. Retail fresh produce sales accounted for 56% of that at $53.6 billion in sales, and food service fresh produce sales accounted for 43% at $41.2 billion in sales.

Ensuring the safety of fresh fruits and vegetables is the top priority for the produce industry. The current state of technology for cleaning food products varies depending on different factors, such as the severity of cleaning standards to be satisfied and type of food products to be cleaned. Generally, conventional cleaning requires a series of steps which attempt to remove particulate contaminant matter from food products. Next, the food products are typically bleached, i.e., subjected to a 100 ppm solution of chlorinated water, and then subjected to further cleaning to remove the chlorinated residue from the surface of food products.

Unfortunately, the conventional food products cleaning processes suffer from several drawbacks. By way of example, large volumes of water are required during a conventional cleaning process. It has been reported that 8800 gallons of water is expended to clean one ton of spinach. Cleaning other types of produce requires similarly high volumes of fresh water.

As another example, chlorine is a toxic substance and is left as a residue on food products after the bleaching step. Additional cleaning is performed and additional equipment is required to remove this residue, which significantly raises the cost of conventional cleaning.

As yet another example, and more importantly, conventional cleaning processes are simply not effective. Food recalls due to the presence of food-borne pathogens are rising and have drastic effects on the food industry and consumers. In 2007, 25 companies recalled over 16,800 tons (>33,750,000 pounds) of food, and in 2008, there have so far been 18 recalls accounting for over 2803 tons of food. In the period between June and July 2008 alone, more than 1000 people were infected by Salmonella saintpaul, leading to a temporary but absolute shut down of the usage of tomatoes in 43 states. Such large-scale food-borne pathogen presence in distributed foods contributes to about 76 million food-borne illness cases in the U.S. each year. For instance, Salmonellosis has been linked to tomatoes, seed sprouts, cantaloupe, watermelon, apple juice, and orange juice. Escherichia coli O157:H7 infection has been associated with lettuce, alfalfa sprouts, and apple juice, and enterotoxigenic E. coli has been linked to carrots. Associations of shigellosis with lettuce, scallions, and parsley, cholera with strawberries, hepatitis A virus with lettuce, raspberries, and frozen strawberries, and Norwalk/Norwalk-like virus with melon, salad, and celery have also been documented. Most recently, Cryptosporidium infection linked to apple cider and cyclospora infection linked to raspberries, lettuce, and basil have broadened awareness that produce-associated illnesses are not confined to bacteria and viruses as causative agents.

Food poisoning outbreaks resulting from ineffective conventional cleaning techniques not only pose a significant health risk to the population at large, but also negatively impact our economy. The Centers for Disease Control and Prevention (CDC) estimates that food poisoning affects 76 million Americans each year, or 25% of the entire U.S. population. The annual costs to treat food borne illness are skyrocketing and are estimated to be from $5 to $6 billion in the U.S. For instance, the costs associated with 10 Salmonella incidents in catering establishments in the U.S. ranged from $57,000 to $700,000, and the “direct cost” of only five Salmonella incidents in manufactured foods ranged from $36,000 to $62 million. Many of the companies involved received enormous publicity but for the wrong reasons, and severe adverse impact on their reputations have put some out of business.

What is therefore needed are improved systems, methods, and compositions for sanitizing food products that do not suffer from the drawbacks encountered by the conventional systems, methods, and compositions for cleaning and sanitizing food products.

### SUMMARY OF THE INVENTION

To achieve the foregoing, the present invention provides systems, methods, and compositions for effectively sanitizing food products using a “charged” solution. A charged (alternatively known as an “activated”) solution is a coherent solution where the distribution of solute particles or molecules are organized in the solvent. Specifically, in a charged solution, the solute particles are arranged in cluster form and exist as solute clusters. In sharp contrast, in conventional cleaning solutions, the solute particles are distributed randomly, and do not exist in any organized fashion.

While not wishing to be bound by theory, the solute clusters in a charged solution of the present invention provide an effective removal and sanitizing mechanism for fine contaminant particles, including microorganisms, from the surface of food products. According to one theory, it is believed that the solute clusters trap the contaminant particles and remove them by the convective flow action of the coherent solution. Solute clusters are believed to bear amphoteric charge which is governed by the solute molecule. Also it is believed that being amphoteric, these solute clusters can effectively target all kinds of particles (including microorganisms) whether they are negatively charged, positively charged or neutral charged microorganism (or bacteria). Thus, in the absence of such solute clusters, as is the case with conventional sanitizing solutions, it is believed that there exists no mechanism to trap such detached particles.

According to another theory, it is believed that the solute clusters in a charged solution carry a sufficiently high amount of charge such that when a solute cluster comes in contact with a microorganism, it collapses the cell wall of the microorganism (i.e., lyses), thereby killing the microorganism.

The present invention recognizes that applying acoustic energy to relatively dilute solutions promotes charging. For example, charging can be accomplished by applying acoustic energy to dilute solutions, where the solute is present in the solvent at a volumetric ration that is between about 1×10⁻³:1 and about 1×10-²⁴:1. Such dilute solutions may cover solute concentrations in the “ultra-dilute regime” and at “near-zero dilutions.” When a solute is present in a solvent at a volumetric ratio that is between about 1×10⁻³:1 and 5×10⁻⁵:1, the dilution of the resulting solution is considered to be in the “ultra-dilute regime.” Furthermore, the term “near-zero dilution,” as used in this specification, refers to dilutions where the solute is present in the solvent at a volumetric ratio that is between about 5×10⁻⁵:1 and 1×10⁻²⁴:1.

Regardless of whether the dilution of the solute is in the “ultra-dilute regime” or at “near-zero dilution,” the teachings of the present invention allow for effective cleaning of food products, without suffering from the drawbacks encountered when using concentrated or dilute conventional sanitizing solutions. In fact, given that relatively dilute concentrations are desirable for charging, i.e., forming solute clusters, solutions having “near-zero dilutions” represent preferred embodiments of the present invention. Use of “near-zero dilutions” for cleaning food products goes against conventional wisdom because conventional sanitizing techniques require higher concentrations of the solute to facilitate particle removal through a reaction mechanism. As explained above, the particle removal mechanism of the present invention is primarily focused on promoting solute cluster formation and not focused on promoting a reaction between the solute and the substrate surface.

In one aspect, the present invention provides an effective method for santizing food products. The method for sanitizing food products includes: (a) activating a solution, which includes a solute and a solvent, by using acoustic energy to form a coherent solution including solute clusters, in which each solute cluster is organized such that at least one solute molecule is surrounded by many solvent molecules; and (b) submerging the food products into a tank containing the coherent solution to sanitize the food products.

Preferably, the solvent includes any one of deionized water or reverse-osmosis water. Acoustic energy may include using at least one of ultrasonic energy or megasonic energy. Preferably, the acoustic energy source has power densities that is between about 1 Watts/cm² and about 8 Watts/cm². The solute may be present in the solvent at a volumetric ratio that is between about 1×10⁻³:1 and about 1×10⁻²⁴:1, is preferably between about 5×10⁻⁵:1 and about 1×10⁻²⁴:1, and is more preferably between about 1×10⁻⁶:1 and about 1×10⁻²⁴:1. Activation may be carried out in the tank in which the food product is submerged.

In alternative embodiments of the inventive methods, activation is carried out in another tank that is different from the tank in which food product is submerged. Acoustic energy used in the present invention may be supplied while food products are submerged in the tank. An effluent stream from the sanitizing tank may be filtered to produce a filtered stream of coherent water which is recirculated back to the tank. Before submerging food products in a coherent solution for sanitizing, coarse particles are removed from the food products.

In another aspect, the present invention provides another method for sanitizing food products. The method for sanitizing food products include: (a) activating a solution, which includes a solute and a solvent, by using acoustic energy to form a coherent solution including solute clusters, in which each solute cluster is organized such that at least one solute molecule surrounded by many solvent molecules; and (b) spraying into a tank droplets of said coherent solution which contact and thereby sanitize food products.

In one embodiment of the present invention, the coherent water solution is conveyed to the tank. According to this embodiment, the food product is preferably sprayed with coherent water using a nozzle, which is equipped with a piezo crystal for generating acoustic energy. Acoustic energy frequencies generated by the piezo crystal may range between about 10 kilohertz and about 3 megahertz. In other embodiments of the inventive methods, the step of activating the solution takes place in a tank separate from the tank in which food products are sprayed. Preferably, the solvent in this embodiment is deionized water or reverse-osmosis water.

In yet another aspect, the present invention provides a system for sanitizing food products. The system includes: (a) a generator for generating coherent solution that includes—(i) a tank capable of holding a solution; and (ii) an acoustic energy source capable of activating the solution to create solute clusters, each of which is organized to include a solute molecule that is surrounded by many solvent molecules; and (b) a recirculation subassembly for recirculating fluid from the tank back to the same tank.

The system provides a recirculation subassembly that includes a filter that comes equipped with UV light source, such that the filter is designed to filter out microorganisms from the effluent stream and the UV light source is capable of killing the microorganisms. In an alternative embodiment, the system of the present invention provides a recirculation subassembly that includes a filter subassembly with a first filter having one or more component filters, each of which is designed to filter out microorganisms and fine particles from the tank. The filter subassembly may also includes a second filter which also has one or more component filters, each of which is designed to filter out microorganisms and fine particles from an effluent stream from the tank, and wherein when the first filter is operational the second filter is not operational and when the second filter is operational the first filter is not operational.

In preferred embodiments, at least some of the component filters in the first and second filters of the present invention connect to a pressure sensor for measuring a back pressure inside the component filters. In this embodiment, the pressure sensor also connects to a first valve subassembly and to a second valve subassembly, such that the first valve subassembly allows flow of the effluent stream in and out of the first filter and second valve subassembly allows flow of effluent stream in and out of the second filter. During operation of the filter subassembly, when the back pressure for a component filter that belongs to the first filter equals or is higher than a first predetermined back-pressure value, then the first valve subassembly is activated to close flow of the effluent stream into the first filter and its component filters and the first valve subassembly opens flow of effluent stream to the second filter and its component filters. Similarly, when the back pressure for a component filter that belongs to the second filter equals or is higher than a second predetermined back-pressure value, then the second valve subassembly is activated to close flow of the effluent stream into the second filter and its component filters and the second valve subassembly opens flow of effluent stream to the first filter and its said component filters.

In one embodiment of the present invention, the first predetermined back-pressure value and a second predetermined back-pressure value are substantially similar. In preferred embodiments, the inventive systems include a filter subassembly with a black-flush subassembly which includes a first back-flush mechanism and a second back-flush mechanism. The first back-flush mechanism capable of providing back-flush water to the first filter and the second back-flush mechanism capable of providing back-flush water to the second filter. During operation of the filter subassembly, when the back pressure for the component filter that belongs to the first valve subassembly equals or is higher than a first predetermined back-pressure value, the first back-flush mechanism may be activated to provide back-flush water to the first filter and its component filters to remove the filtered microorganisms and fine particles from the first filter and its component filters. Similarly, when the back pressure for a component filter that belongs to the second valve subassembly equals or is higher than a second predetermined back-pressure value, then the second back-flush mechanism is activated to provide back-flush water to the second filter and its component filters to remove the filtered microorganisms and fine particles from the second filter and its component filters.

In other embodiments of the present invention, the system includes a UV radiation chamber which is capable of receiving and destroying the microorganisms removed from the first filter and from the second filter.

The inventive systems may include a first filtered line designed to convey a first filtered stream generated from the first filter; a second filtered line designed to convey a second filtered stream generated from the second filter; and wherein each of the first and second filtered lines connect to the tank to convey the first and said second filtered streams from respective first and second filters to the sanitizing tank. The inventive systems may also include a UV-treated back-flush effluent line for conveying a stream of UV-treated back-flush effluent stream generated from the UV radiation chamber; a particle filter designed to remove dead microorganisms from the UV-treated back-flush effluent stream; and wherein said UVtreated back-flush effluent line connects the UV radiation chamber to the particle filter.

In yet another aspect, the present invention provides a composition for sanitizing food products. The composition for sanitizing includes: (a) sodium chloride in effective amounts to form a coherent solution; (b) a solvent that includes any one of deionized water and reverse osmosis water; and (c) wherein the sodium chloride is present in effective amounts in the solvent so that when sufficient amounts of acoustic energy are supplied to the sodium chloride and the solvent, a coherent solution is produced in which the sodium chloride exists as clusters such that each said sodium chloride cluster is organized to include at least one sodium chloride molecule which is surrounded by many solvent molecules. Preferably, in the compositions of the present invention, sodium chloride is present in the solvent at a volumetric ratio that is between about 1×10⁻³:1 and about 1×10⁻²⁴:1. More preferably, sodium chloride is present in the solvent at a volumetric ratio that is between about 5×10⁻⁵:1 and about 1×10⁻²⁴:1, and even more preferably, sodium chloride is present in the solvent at a volumetric ratio that is between about 1×10⁻⁶:1 and about 1×10⁻²⁴:1. The acoustic energy source, preferably a megasonic device, has power densities that may be equal to or higher than about 3 Watts/cm².

The method of operation of the invention, however, together with additional objects and advantages thereof will be best understood from the following descriptions of specific embodiments when read in connection with the accompanying figures.

### BRIEF DESCRIPTION OF THE FIGURES

FIG. 1 shows a system, according to one embodiment of the present invention, for sanitizing food products.

FIG. 2 shows another system, according to an alternative embodiment of the present invention, for sanitizing food products.

FIG. 3 shows a filter subassembly, according to one embodiment of the present invention, which is preferably integrated into the system of FIG. 1.

FIG. 4 shows solute particles arranged in cluster form in a coherent solution, according to one embodiment of the present invention.

### DETAILED DESCRIPTION OF THE INVENTION

In the following description numerous specific details are set forth in order to provide a thorough understanding of the present invention. It will be apparent, however, to one skilled in the art that the present invention may be practiced without limitation to some or all of these specific details. In other instances, well-known process steps have not been described in detail in order to not unnecessarily obscure the invention.

The present invention provides systems, methods, and compositions for sanitizing food products using relatively low-concentrations of a solute, such as sodium chloride (i.e., salt) in a coherent solution. In the coherent solution, the solute exists as a solute cluster where at least one solute particle or molecule is surrounded by many solvent molecules. The present invention also provides systems, methods, and compositions which facilitate filtering and recycling the coherent solution after sanitizing for subsequent use in the inventive sanitizing processes. As a result, the ability of the present invention to conserve water during sanitizing represents a marked improvement over the conventional techniques of cleaning produce.

In the present invention, relatively low solute concentrations are preferred in certain embodiments because they better lend themselves to charging. While wishing not to be bound by theory, it is believed that in a charged solution, solute particles, which are arranged as clusters, facilitate detaching fine contaminant particles (including microorganisms) from the food product's surface and then facilitate trapping the detached particles for effective removal from the food product. As a result, the present invention focuses on forming solute clusters for effective cleaning of food products. Notably, the inventive cleaning systems and methods described herein not only sanitize food products using relatively low solute concentrations, which are deemed ineffective in conventional cleaning, rather in certain instances, such low solute concentrations represent preferred embodiments of the present invention.

Relatively low solute concentrations are also preferred because they promote more effective filtration of effluent streams to allow for recycling the coherent solution during sanitizing operations. A higher solute concentration is undesired as it may frequently clog or damage filters. In sharp contrast, low solute concentration of the present invention circumvents such drawbacks.

The recycling feature of the present represents a marked improvement over the prior art. By way of example, by repeatedly recycling the coherent water of the present invention during the sanitization process, numerous sanitizing cycles can be performed without requiring high volumes of fresh water, as is necessary when implementing conventional sanitizing techniques.

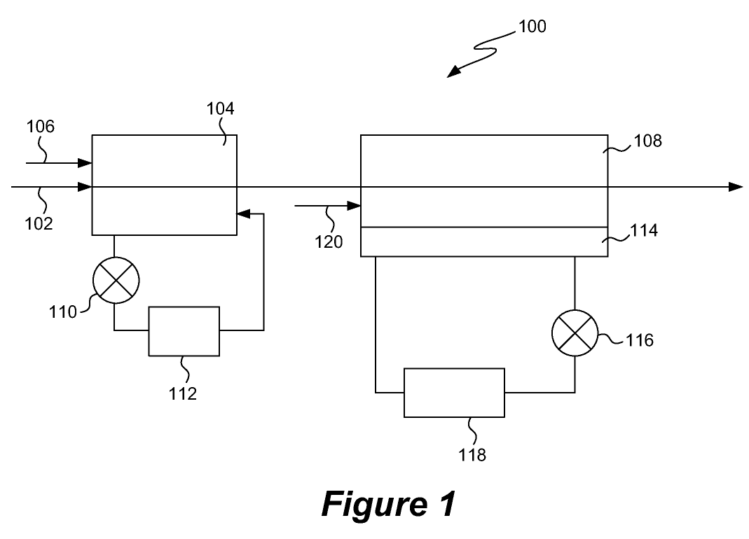

FIG. 1 shows a cleaning and sanitizing system 100, according to one embodiment of the present invention, for effectively cleaning and sanitizing food products. System 100 has a coarse cleaning tank 104 for removing coarse particulate contaminants from food products and a sanitizing tank 108 for removing fine particulate contaminants (including microorganisms) from food products. A stream of tap water 106 is introduced inside tank 104 for implementing coarse particle removal. For fine particle removal and sanitizing food products, a stream of low-concentration solution 120 is introduced into tank 108. Tank 108 is fitted with an acoustic energy source (i.e., an ultrasonic device or a megasonic device) to activate or charge the low-concentration solution residing in tank 108 to form a coherent solution for sanitizing food products in tank 108.

A conveyance mechanism 102 (e.g., a conveyor belt assembly) connects tanks 104 and 108. Specifically, as shown in FIG. 1, mechanism 102 transports food products into and out of coarse cleaning chamber 104 and then into and out of sanitizing chamber 108. Each tank has associated with it a pump for evacuating the contents inside that tank. By way of example, pump 110 evacuates the contents of tank 104 and advances them to a coarse-filtration subassembly 112, which is preferably a waste-water treatment plant. Similarly, pump 116 evacuates the contents of tank 108 and advances them to a fine-filtration subassembly 118, which traps fine particles including microorganisms. Fine-filtration subassembly 118 preferably includes a UV radiation source to kill the microorganisms present in effluent of tank 108. Inside subassembly 118 and downstream from the UV radiation source, a particle filter may be disposed to remove fine particles, including dead microorganisms, from the effluent of tank 108.

Of particular interest is a recycling loop or closed path of the coherent solution that is defined by the different connections that link tank 108, pump 116 and filtration assembly 118. Coherent solution may pass through this loop once or numerous times during a sanitization process depending on when the coherent solution has acceptable reduced levels of fine particles (including microorganisms). In preferred embodiments of the present invention, sample ports are provided at various locations along the recycling loop to obtain samples of coherent solution, which are analyzed to determine whether the recycling loop has sufficiently reduced levels of fine particles (including microorganisms). Upon accomplishing acceptable low levels of fine particle counts, conveyance mechanism 102 advances the food products from tank 108 to another location where they may be ready for packaging or drying.

Tanks 104 and 108 can be any rigid body which can hold water and food products. Tank 104 is designed to remove coarse particles, which refers to particles having a dimension that is greater than about 500 microns. Fine particles, on the other hand, are those that have their largest dimension range from between about 1 micron and about 20 microns.

Acoustic energy source 114 can be any device (e.g., ultrasonic or megasonic) that introduces acoustic energy inside coherent water. In preferred embodiments of the present invention, source 114 is a megasonic device as it effectively transforms a relatively dilute solution from an uncharged state to a charged state (i.e., coherent solution). Although megasonic outputs as high as 5 Watts/cm² and higher may work well, it is possible to that outputs of about 3 Watts/cm² and lower may provide the requisite energy for charging. Suitable equipment for generating megasonic energy is commercially available from a variety of vendors. Such equipment is commercially available and typically includes a generator and a series of special transducers or the like. By way of example, megasonic devices, which are commercially available from Kaijo Corporation of Japan, PCT Systems, Inc. of Fremont, Calif., and ProSys Incorporation of Campbell, Calif., work well.

In preferred embodiments where sanitizing chamber 108 is equipped with an acoustic energy source, sanitizing chamber 108 can be made of any material known to be a good transmitter of acoustic energy. These chambers are preferably made from quartz. In one embodiment, sanitizing chamber is ideally placed above its corresponding acoustic energy source.

The solvent in sanitizing chamber 108 is preferably purified water, (e.g., deionized or reverse osmosis water). The solute is preferably sodium chloride, but other solutes may also work well. By way of example, solutions that include other solutes, such as ammonia, hydrogen peroxide or other types of salts are also effective. In the charged solution, a volumetric ratio of the solute to solvent is between about 1×10⁻³:1 and about 1×10⁻²⁴:1, preferably between about 5×10⁻⁵:1 and about 1×10⁻²⁴:1, and more preferably between about 1×10⁶:1 and about 1×10⁻²⁴:1.

In certain embodiments, the present invention can be designed to sanitize one or more food products in each cycle. Moreover, those skilled in the art will recognize that in certain embodiments, each food product can be put through multiple sanitizing cycles and at varying concentrations of charged solution so as to provide food products that have a desired low level of fine particle count on their surface and which are substantially free of contaminant microorganisms.

FIG. 1 shows megasonic 114 incorporated into sanitizing tank 108. In other preferred embodiments, however, the megasonic device need not be so incorporated and can be installed in a separate tank where a charged solution is prepared (outside tank 108) and then introduced into tank 108 through stream 120. This embodiment allows use of large megasonic power or energies to charge the low-concentration solution, without affecting the quality of produce.

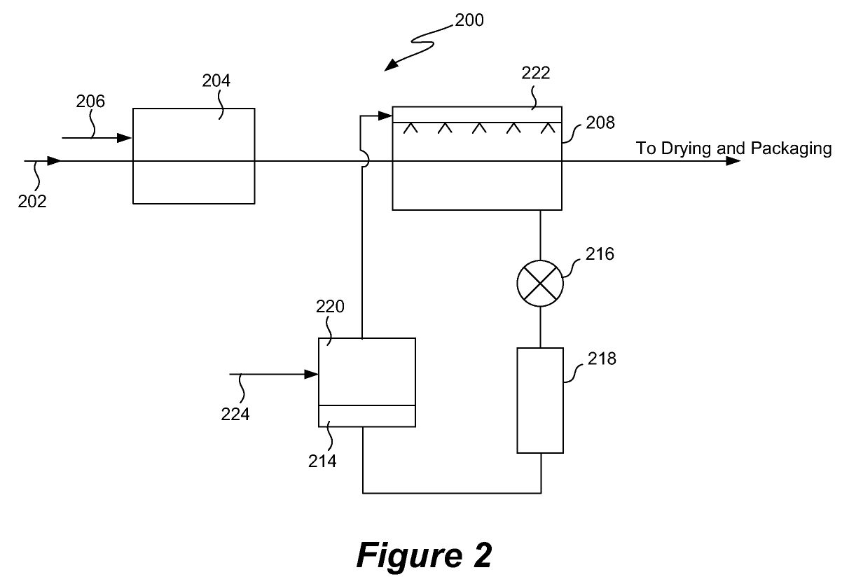

FIG. 2 shows a cleaning and sanitizing system 200, according to an alternative embodiment of the present invention, for effectively cleaning and sanitizing food products. System 200 includes a coarse-cleaning tank 204, tap water stream 206, and conveyance mechanism 202, which are substantially similar to tank 104, stream 106 and conveyance mechanism (i.e., preferably a conveyor belt) 102 of FIG. 1, respectively. System 200 also includes a pump and coarse filtration subassembly (not shown to simplify illustration), that are substantially similar to pump 110 and coarse filtration subassembly 112. Like coarse filtration subassembly 104, coarse filtration subassembly of system 200 is preferably a waste-water treatment plant.

System 200 shows a sanitizing chamber 208 fitted with a spray mechanism 222 that is designed to spray coherent water on food products (placed on conveyance mechanism 202). In one embodiment of the present invention, spray mechanism 222 comprises a nozzle which includes piezo-electric crystals to acoustically charge the solution before it is sprayed. In addition to the spray feature, system 200 also differs from system 100 of FIG. 1 with respect to where the coherent solution is formed. In system 200, a separate charging tank 220 is provided for generating a charged solution (which is a coherent solution). The charged solution is introduced into tank 208 for sanitizing food products.

In one embodiment of the present invention, a stream of solution, which includes a solute and a solvent, in an uncharged state is provided inside tank 220. Charging tank 208 is equipped with an acoustic energy source 214, preferably a megasonic device, to provide the requisite megasonic energy to charge the solution held inside tank 220, which is subsequently advanced for sanitizing food products.

Charging tank 220 is a rigid body designed to hold sanitizing solution 224 and can be made of any material known to be a good transmitter of acoustic energy. Such chambers are preferably made from quartz. Acoustic energy source 214 is substantially similar to acoustic energy source 114 of FIG. 1. In this preferred embodiment, because the charging chamber is separate from the sanitizing chamber, higher frequencies of megasonic energy may be used to create “super-charged” solution more effective for sanitizing food products without any concern for damaging them.

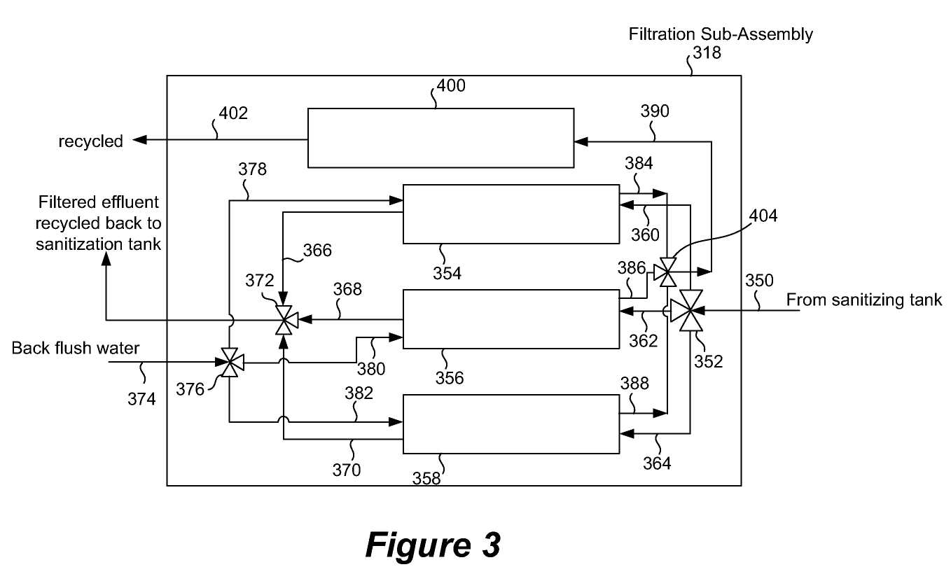

Pump 216 evacuates the contents (except food products) inside sanitizing chamber 208 and conveys them to fine filtration subassembly 218, which removes fine particulates (including dead microorganisms) from the contents. As a result, subassembly 218 recycles coherent solution back to the sanitizing tank 208. In preferred embodiments of the present invention, subassembly 118 and 218 are substantially similar to the fine filtration subassembly 318 shown in FIG. 3.

FIG. 3 shows a filtration subassembly 318, according to one embodiment of the present invention. Subassembly 318 is designed to effectively filter an effluent stream 350 from a sanitizing tank (e.g., tank 108 of FIG. 1 and tank 208 of FIG. 2). According to FIG. 3, effluent stream 350 is guided into the appropriate filter(s) through a valve assembly. Specifically, a three-way valve 352 facilitates introduction of the effluent stream into component filter 354 through line 360, into component filter 356 through line 362 and into component filter 358 through line 364. In the embodiment shown in FIG. 3, filter components 354 and 356 comprise a first filter and filter 358 functions as a second filter. Each of first and second filters may include one or more component filters. Regardless of the number of component filters employed in a particular filter, when the first filter is operational, the second filter is non-operational and vice versa. During the non-operational state of a filter, subassembly 318 facilitates cleaning of that filter using back-flush water, as will be explained later.

Recycling is an important feature in preferred embodiments of the present invention. In such embodiments, a filtered stream resulting from a component filter is recycled back to the sanitizing tank for further use. By way of example, in FIG. 3, filtered stream from components 354, 356, and 358 flows through lines 366, 368, and 370, respectively, and through a three-way valve 372 so that it can be recycled back into a sanitizing tank.

A back-flush feature of subassembly 318 is another important feature of the preferred embodiments of the present invention. Each component filter is fitted with a pressure sensor (not shown to simplify illustration) for sensing the back pressure buildup in the component filter during a filtration process. The pressure sensor is connected (also not shown) to valve assemblies 352 and 372 such that when the back pressure in a particular component filter equals or exceeds a predetermined threshold value, the effluent stream access to that particular component filter and its associated component filter is closed and the back-flush water access to that component filter and its associated component filters is initiated to remove fine particulate contaminants (including microorganisms) from the filter (which includes that component and its associated filters). By way of example, if the back-pressure build up in component filter 354 equals or exceeds a predetermined back pressure value, then valve 352 closes effluent flow into component filters 354 and 356 as they comprise first filter. In reference to FIG. 3, back-flush water is introduced into component filters 354, 356, and 358 through a three-way valve 376 through lines 378, 380, and 382, respectively. Furthermore, another three-way valve 404 is activated to allow the effluent back flush water from component filters 354, 356, and 358 to be advanced through lines 384, 386, and 388 to a single line 390 which connects to a filter chamber 400. Chamber 400 comes fitted with a UV radiation source to kill microorganisms present in the back-flush effluent stream and remove particulate matter, including dead microorganisms, from this stream. After the particulate matter is removed, the filtered back-flush stream in preferred embodiments of the present invention is recycled to either coarse or fine particle removal tanks.

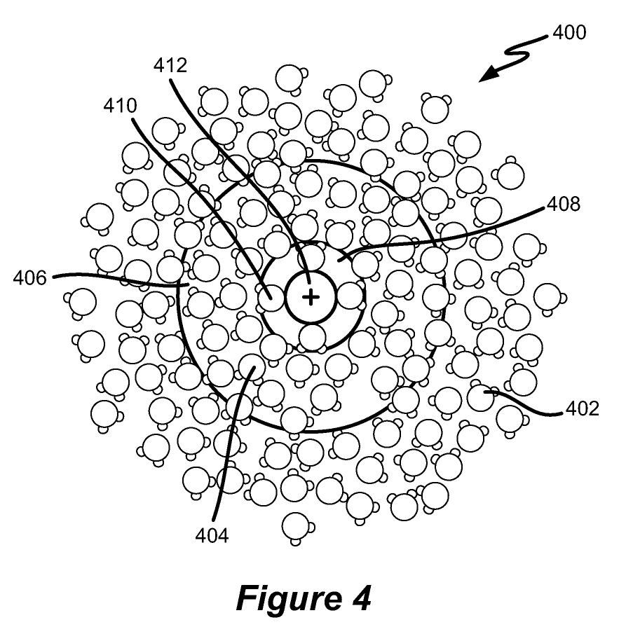

FIG. 4 shows the distribution of solute molecules in an activated or charged state. In a charged solution, a solute molecule 412 is organized as a single solute cluster 400. In each solute cluster 400, typically one solute molecule 412 is surrounded by many solvent molecules. Inside the inner hydration shell, which represents a region that is the closest to solute molecule 412, solvent molecules 410 surround the solute molecule and are present in a highly ordered fashion as shown in FIG. 4. In an outer hydration shell 406, solvent molecules 404 which surround solute molecule 412, are semi-ordered. In other words, solvent molecules 404 are relatively less ordered than the solvent molecules 410 in solvent area 408. Outside the outer hydration shell 406, there is greater disorder of the solvent molecules than outside solvent area 408. Well outside outer hydration shell 406 bulk water conditions exist. Specifically, solvent molecules 402 which surround solute molecule 412 are randomly distributed and have very little or no order. In fact, in non-activated or non-charged solutions, solvent molecules are randomly distributed as shown by solvent molecules 402 in FIG. 4, and solvent molecules are not arranged in an ordered fashion or have no particular orientation around a solute molecule like either solvent molecules 404 or solvent molecules 408.

In accordance with one embodiment of the present invention, a sanitization process using such solute clusters begins when food products are cleaned to remove coarse particles. Specifically, food products are placed on a conveyance mechanism are advanced to a coarse-particle removal tank (e.g., tank 104 of FIG. 1). A stream of water (e.g., stream 106 of FIG. 1) is provided in the coarse-particle removal tank to remove coarse particles from the surface of food products. The contents (not including the food products) inside this tank are evacuated by a pump (e.g., pump 110) and sent to a coarse filtration subassembly (e.g., subassembly 112 of FIG. 1), which removes coarse particles from the water. The resulting stream of filtered water is recycled back to the tank for further coarse particle removal. In preferred embodiments, coarse-filtration subassembly comprises a waste-water treatment plant for effecting removal of coarse particles from the effluent of the coarse-particle removal tank. In this manner, water may be recycled from the coarse-particle removal tank and coarse-filtration subassembly multiple times to make sure that the food products are free of coarse particles.

Preferably contemporaneous with the coarse-particle removal step described above, a solution (having a solute and a solvent), preferably of low solute concentration, is introduced inside fine-particle removal tank (e.g., stream 120 is introduced inside tank 108 of FIG. 1). In this tank, the solution is subjected to a requisite amount of acoustic energy from an acoustic source (e.g., acoustic source 114 of FIG. 1) such that the solution is charged. A charged solution according to the present invention is a coherent solution which includes solute clusters (e.g., cluster 400 of FIG. 4). As shown in FIG. 4, solute clusters have a solute particle or molecule surrounded by many solvent molecules.

When the coarse particle removal has concluded, the conveyance device advances the food products into a sanitizing tank (e.g., tank 108 of FIG. 1), which may be filled with coherent solution. In the sanitizing tank, the food products are ultimately submerged into the coherent solution so that microorganisms are killed. In certain preferred embodiments of the present invention, a holding tank might be used to prepare and store some of the coherent solution which is conveyed to the sanitizing tank when the food products arrive. Regardless of how a coherent solution is provided in the sanitizing tank, food products should be submerged in the coherent solution for sufficiently long periods of time to effect sanitizing of food products. Food products are preferably submerged in coherent solution for a period of time that is between about 1 minute and about 5 minutes.

While not wishing to be bound by theory, it is believed that more than one mechanism may be involved in killing microorganisms present on the surface of food products. According to one theory, the solute clusters inside the coherent solution act as scavengers—i.e., solute clusters attract and trap fine particles including microorganisms. According to another theory, if the solute clusters carry a sufficiently large amount of charge, then upon contact the clusters lyse the microorganisms.

The effluent from the sanitizing tank is then pumped (e.g., using pump 116 of FIG. 1) into a fine-filtration subassembly (e.g., subassembly 118 of FIG. 1) which removes fine particles including dead or living microorganisms. The fine-filtration assembly may include a UV radiation source which kills any living microorganisms. It is believed that as the coherent solution passes through the fine-filtration subassembly it retains at least some of its charged state. The filtered coherent stream leaves this assembly and is recycled back to the sanitizing tank for further charging and sanitizing of food products.

In this manner, coherent solution may make multiple passes through the recycle loop between sanitizing tank and fine-filtration subassembly until the food products are substantially free of microorganisms and the fine particle count is below a desired level. Samples of the coherent solution at various locations along the recycle loop may be taken to ensure that food products are substantially free of microorganisms or that the fine particle count is below a desired level. After removal of fine particles (including microorganisms) concludes, then the conveyance mechanism advances food products for drying and packaging.

In certain preferred embodiments of the present invention, the filtered coherent solution exiting the fine-filtration subassembly may go to a holding tank which is equipped with an acoustic energy source and specifically designed to initially charge a solution and also designed to further charge a filtered coherent solution. In other preferred embodiments of the present invention, a mixing chamber which mixes a solute (e.g., salt) and a solvent (e.g., deionized or reverse-osmosis water) may advance the solution to this holding tank.

After coarse particle removal, instead of submerging food products in a coherent solution (as shown in FIG. 1), it is also possible to sanitize food products by spraying them with the coherent solution, as shown in FIG. 2. While not wishing to be bound by theory, it is believed that in this embodiment that by charging the solution to a greater extent in a tank (e.g., tank 220) separate from the sanitizing tank (e.g., tank 208), the charged solution can lyse microorganisms. Thus, the embodiment shown in FIG. 2 has the advantage of allowing transfer of relatively higher levels of acoustic energy (that would normally damage the food product), resulting in “super-charged” solution that is more potent in removing fine particulate contaminants from the food product and also killing the microorganisms.

Fine filtration in either the embodiment of FIG. 1 or of FIG. 2 can be carried out using the fine-filtration subassembly shown in FIG. 3. In one preferred embodiment of the present invention, a filtration process begins when an effluent stream is conveyed using a pump, (e.g., pump 116 of FIG. 1 or 216 of FIG. 2) from a sanitizing tank (e.g., 108 of FIG. 1 or 208 of FIG. 2) to a filtration subassembly, such as subassembly 318 shown in FIG. 3. Inside the filtration subassembly, the effluent stream is delivered into a first filter (e.g., filter components 354 and 356 of FIG. 3) or to a second filter (e.g., filter component 358 of FIG. 3). Depending on the filter component(s) that are selected to effect filtration, the resulting filtered effluent stream flows either through a first filtration line (e.g., lines 366 and 368 of FIG. 3) or through a second filtration line (e.g., line 370 of FIG. 3), ultimately flowing back to the same sanitization tank or to another process. The fact that the present invention uses low concentration of solute in a coherent solution for sanitizing and fine particle removal, the coherent solution lends itself to being easily recycled and used again in the cleaning process. Furthermore, as the coherent water passes through the filters, various lines, valves, and/or pumps, it retains at least some of the solute clusters generated during the charging or activating step described in connection with FIGS. 1 and 2. As a result, some of the energy expended to create solute clusters is recovered and used again for cleaning. The ability of the present invention to conserve both energy and water is an important renewable feature which represents a marked improvement over the prior art.

During the filtration process, particulate matter (including microorganisms) have a tendency to get trapped inside component filters and build up undesired back pressure inside the component filters. Significant particulate build up impedes the ability of the component filters to effectively carry out filtration of the effluent stream from the sanitizing tank and may ultimately cause damage to the component filter. As a result, during filtration, a back pressure of the component filter is measured and monitored. If the back pressure inside a component filter builds up to reach or exceed predetermined levels, then a mechanism to clean out the particulate build up is initiated. Specifically, the effluent stream supply from the sanitizing tank to a filter (which includes that component filter) is shut off and a back-flush water stream is introduced into that filter to flush out the particulate buildup. The resulting back-flush effluent stream from the filter is advanced to a filtration chamber, which in preferred embodiments of the present invention comes equipped with a UV radiation source to kill microorganisms inside this effluent stream. A filtered back-flush water stream, substantially free of particulate matter and microorganisms, is preferably recycled back to the sanitizing tank.

As described above, a filter, which included a component filter plugged with particulate build up due to filtration over time, is cleaned and prepared for subsequent filtering of effluent from the sanitizing tank while another filter carries out the filtration process. Consequently, the back-flush feature of the preferred embodiments of the present invention allows filtration of the effluent stream from the sanitizing tank to be carried out in batch or continuous mode of operation. The ability of the present invention to provide uninterrupted filtration of the effluent from the sanitizing tank also allows the sanitizing process of the present invention to realize a high throughput.

Although illustrative embodiments of this invention have been shown and described, other modifications, changes, and substitutions are intended. For example, the charging tank, in which solute clusters are generated, a recirculation scheme is preferably included to recirculate the solution undergoing charging. This recycling feature provides both a greater degree of charging (i.e., forms a greater number of solute clusters) and facilitates uniform distribution of the solute, which is present in low concentrations, inside the charging tank. For these and other reasons, it is appropriate that the appended claims be construed broadly and in a manner consistent with the scope of the disclosure, as set forth in the following claims.

What is claimed is:

1. A method for sanitizing food products, comprising:
activating a solution, which includes a solute and a solvent, by using acoustic energy to form a coherent solution including solute clusters, in which each solute cluster is organized such that at least one solute molecule is surrounded by many said solvent molecules; and
submerging said food products into a tank containing said coherent solution to sanitize said food products.

2. The method of claim 1, wherein said solvent includes any one of deionized water and reverse osmosis water.

3. The method of claim 1, wherein said acoustic energy includes using at least one of ultrasonic energy or megasonic energy.

4. The method of claim 3, wherein said megasonic has power densities that are between about 1 Watts/cm² and about 8 Watts/cm².

5. The method of claim 1, wherein in said coherent solution, said solute is present in said solvent at a volumetric ratio that is between about 1×10⁻³:1 and about 1×10⁻²⁴:1.

6. The method of claim 5, wherein said solute is present in said solvent at a volumetric ratio that is between about 5×10⁻⁵:1 and about 1×10⁻²⁴:1.

7. The method of claim 6, wherein said solute is present in said solvent at a volumetric ratio that is between about 1×10⁻⁶:1 and about 1×10⁻²⁴:1.

8. The method of claim 1, wherein said activating is carried out in said tank.

9. The method of claim 1, wherein said activating is carried out in another tank that is different from said tank.

10. The method of claim 1, further comprising supplying acoustic energy during said submerging of said food products.

11. The method of claim 1, wherein an effluent stream from said tank is filtered to produce a filtered stream of coherent water.

12. The method of claim 1, wherein said coherent stream is recirculated back to said tank.

13. The method of claim 1, further comprising cleaning said food products to remove coarse particles therefrom before said submerging said food products.

14. A method for sanitizing food products, comprising:
activating a solution, which includes a solute and a solvent, by using acoustic energy to form a coherent solution including solute clusters, in which each solute cluster is organized such that at least one solute molecule surrounded by many solvent molecules;
spraying into a tank droplets of said coherent solution which contact and thereby sanitize food products.

15. The method of claim 14, further comprising conveying to said tank said coherent water solution.

16. The method of claim 14, wherein said spraying is accomplished using a nozzle which is equipped with a piezo crystal for generating acoustic energy.

17. The method of claim 16, wherein said piezo crystal generates frequencies which range between about 10 kilohertz and about 3 megahertz.

18. The method of claim 14, further comprising activating said solution in another tank different from said tank.

19. The method of claim 14, wherein said solvent is any one of deionized water or reverse osmosis water.

20. A system for cleaning food products, comprising:
a generator for generating coherent solution including:
a tank capable of holding a solution;
an acoustic energy source capable of activating said solution to create solute clusters each of which is organized to include a solute molecule that is surrounded by many solvent molecules;
a recirculation subassembly for recirculating fluid from said tank back to said tank.

21. The system of claim 20, wherein said recirculation subassembly includes a filter that comes equipped with UV light source, said filter is designed to filter out microorganisms and fine particles from effluent stream and said UV light source is capable of killing said microorganisms.

22. The system of claim 20, wherein said recirculation subassembly further comprising a filter subassembly which includes a first filter having one or more component filters, each of which is designed to filter out microorganisms and fine particles from said tank.

23. The system of claim 22, said filter subassembly further comprising a second filter which includes one or more component filters, each of which is designed to filter out microorganisms and fine particles from an effluent stream from said tank, and wherein when said first filter is operational said second filter is not operational, and when said second filter is operational said first filter is not operational.

24. The system of claim 23, wherein at least some of said component filters in said first and said second filters connect to a pressure sensor for measuring a back pressure inside said component filters and said pressure sensor is connected to a first valve subassembly and a second valve subassembly, said first valve subassembly allows flow of said effluent in and out of said first filter, and second valve subassembly allows flow of effluent in and out of said second filter, such that during operation of said filter subassembly, when said back pressure for a component filter that belongs to said first filter equals or is higher than a first predetermined back-pressure value, then said first valve subassembly is activated to close flow of effluent stream into said first filter and its component filters, and said first valve subassembly opens flow of effluent stream to said second filter and its said component filters, and when said back pressure for a component filter that belongs to said second filter equals or is higher than a second predetermined back-pressure value, then said second valve subassembly is activated to close flow of effluent stream into said second filter and its component filters, and said second valve subassembly opens flow of effluent stream to said first filter and its said component filters.

25. The system of claim 24, wherein said first predetermined back-pressure value and said second predetermined back-pressure value are substantially similar.

26. The system of claim 24, wherein said filter subassembly further comprises a back-flush subassembly which includes a first back-flush mechanism and a second back-flush mechanism, said first back-flush mechanism is capable of providing back-flush water to said first filter and said second back-flush mechanism is capable of providing back-flush water to said second filter.

27. The system of claim 26, wherein during operation of said filter subassembly, when said back pressure for said component filter that belongs to said first valve subassembly equals or is higher than a first predetermined back-pressure value, then said first back-flush mechanism is activated to provide back-flush water to said first filter and its said component filters to remove the filtered microorganisms and fine particles from said first filter and its component filters and when said back pressure for a component filter that belongs to said second valve subassembly equals or is higher than a second predetermined back-pressure value, then said second back-flush mechanism is activated to provide back-flush water to said second filter and its said component filters to remove the filtered microorganisms and fine particles from said second filter and its said component filters.

28. The system of claim 27, further comprising a UV radiation chamber which is capable of receiving and destroying said microorganisms removed from said first filter and from said second filter.

29. The system of claim 24, further comprising:
a first filtered line designed to convey a first filtered stream generated from said first filter;
a second filtered line designed to convey a second filtered stream generated from said second filter; and
wherein each of said first and said second filtered lines connect to said tank to convey said first and said second filtered streams from respective said first and second filters to said tank.

30. The system of claim 28, further comprising:
a UV-treated back-flush effluent line for conveying a stream of UV-treated back flush effluent generated from said UV radiation chamber;
a particle filter designed to remove dead microorganisms from said stream of UV-treated back-flush effluent; and
wherein said UV-treated back-flush effluent line connects said UV radiation chamber to said particle filter.

31. A composition for sanitizing food products, said composition comprising:
sodium chloride in effective amounts to form a coherent solution;
a solvent that includes any one of deionized water or reverse osmosis water; and
wherein said sodium chloride is present in effective amounts in said solvent so that when sufficient amounts of acoustic energy are supplied to said sodium chloride and said solvent, a coherent solution is produced in which said sodium chloride exists as clusters such that each said sodium chloride cluster is organized to include at least one sodium chloride molecule which is surrounded by many solvent molecules.

32. The composition of claim 31, wherein said sodium chloride is present in said solvent at a volumetric ratio that is between about 1×10⁻³:1 and about 1×10⁻²⁴:1.

33. The composition of claim 32, wherein said sodium chloride is present in said solvent at a volumetric ratio that is between about 5×10⁻⁵:1 and about 1×10⁻²⁴:1.

34. The composition of claim 33, wherein said sodium chloride is present in said solvent at a volumetric ratio that is between about 1×10⁻⁶:1 and about 1×10⁻²⁴:1.

35. The composition of claim 31, wherein said megasonic has power densities that are between about 1 Watts/cm² and about 8 Watts/cm².
